(19) United States
(12) Patent Application Publication
(10) Pub. No.: US 2024/0189374 A1
(43) Pub. Date: Jun. 13, 2024

(54) ADENOVIRUS FOR TREATMENT OF CANCER

(71) Applicant: ELICERA THERAPEUTICS AB, Göteborg (SE)

(72) Inventor: Di YU, Storvreta (SE)

(21) Appl. No.: 18/556,408

(22) PCT Filed: Apr. 21, 2022
(86) PCT No.: PCT/SE2022/050389

§ 371 Date: Oct. 20, 2023

(30) Foreign Application Priority Data
Apr. 23, 2021 (SE) 2150511-0

(51) Int. Cl. A61K 35/761; A61K 38/00; A61P 35/00; C07K 14/195; C07K 14/525; C12N 15/86

(52) U.S. Cl. CPC A61K 35/761; A61P 35/00; C07K 14/195; C07K 14/525; C12N 15/86; A61K 38/00; C12N 2710/10022; C12N 2710/10032; C12N 2710/10043; C12N 2710/10052; C12N 2710/10071

(57) ABSTRACT

The invention relates to an adenovirus comprising a nucleic acid sequence encoding a Helicobacter pylori neutrophil-activating protein (NAP) and/or a nucleic acid sequence encoding an immunologically equivalent fragment of NAP and a nucleic acid sequence encoding an immunomodulator capable of inducing an immune response in a subject. The adenovirus has enhanced clinical effects in terms of delaying tumor growth and prolonging survival.

### TECHNICAL FIELD

The present invention generally relates to adenoviruses, and in particular to recombinant adenoviruses useful in treatment of cancers.

### BACKGROUND

Current cancer treatments are mainly based on chemotherapy, radiotherapy and/or surgery. In spite of an elevated rate of cure for cancer at early stages, many advanced cancers are incurable because they cannot be eradicated surgically or because the doses of radiotherapy or chemotherapy administered are limited by their toxicity in normal cells.

Virotherapy using oncolytic viruses have been proposed as an alternative or complement to traditional cancer treatments. Viriotherapy uses an oncolytic virus capable of replicating and propagating selectively in tumor cells. These oncolytic viruses thereby selectively infect and lyse tumor cells where after the released progeny virions re-infect neighboring tumor cells, and may also enter the blood stream to infect metastasized tumor cells.

Virotherapy using oncolytic viruses should meet two main requirements; selectivity and potency. Different strategies have been proposed to obtain selectivity towards tumor cells, including elimination of viral functions that are necessary for replication in normal cells but that are not needed in tumor cells, the control of the viral genes that start the replication using tumor-selective promoters and the modification of the virus capsid proteins implied in the infection of the host cell.

Antitumor immune response mounted by oncolytic viruses, however, generally seems to be insufficient to acquire a good therapeutic effect in clinical settings, i.e., too poor potency. Accordingly, insertion of therapeutic genes into the genome of the oncolytic viruses has been suggested to boost their potency. Various such therapeutic genes have been proposed in the art, including activation of a prodrug with bystander effect, activation of the immune system against the tumor, induction of apoptosis, and inhibition of angiogenesis.

Ramachandran et al., An infection-enhanced oncolytic adenovirus secreting H. pylori neutrophil-activating protein with therapeutic effects on neuroendocrine tumors, Molecular Therapy (2013) 21(11): 2008-2018 and Ramachandran et al., Vector-encoded Helicobacter pylori neutrophil-activating protein promotes maturation of dendritic cells with Th1 polarization and improved migration, The Journal of Immunology (2014) 193(5): 2287-2296 disclose a replication-selective, infection-enhanced adenovirus with secretory neutrophil-activating protein (NAP). NAP adenovirus promoted maturation and migration of dendritic cells (DCs) and improved median survival in mice. Zhang et al., Recombinant adenovirus expressing a soluble fusion protein PD-1/CD137L subverts the suppression of CD8+ T cells in HCC″, Molecular Therapy (2019) 27(11): 1906-1918 discloses that the recombinant adenovirus induced a tumor-specific and systemic protection against tumor re-challenges.

There is still a need for improving the potency of oncolytic viruses.

### SUMMARY

It is a general objective of the invention to provide an adenovirus having therapeutic effect in treatment of cancer.

This and other objectives are met by embodiments disclosed herein.

The present invention is defined in the independent claims. Further embodiments of the invention are defined in the dependent claims.

An aspect of the invention relates to an adenovirus comprising a nucleic acid sequence encoding a Helicobacter pylori neutrophil-activating protein (NAP) and/or a nucleic acid sequence encoding an immunologically equivalent fragment of NAP. The immunologically equivalent fragment of NAP is a fragment including at least one polypeptide domain of at least 20 amino acid residues of NAP The adenovirus also comprises a nucleic acid sequence encoding an immunomodulator capable of inducing an immune response in a subject.

Further aspects of the invention relates to an adenovirus according to above for use as a medicament and for use in treatment of cancer.

The recombinant adenoviruses of the embodiments have enhanced therapeutic effect in terms of significantly inhibiting tumor growth and significantly prolonging survival in subjects following tumor implantation. This enhanced therapeutic effect is achieved by engineering the adenovirus to co-express NAP, and/or an immunologically equivalent fragment thereof, and an immunomodulator. The combination of NAP and the immunomodulator made the adenovirus capable of inducing immunogenic cell death in cancer cells, and in addition induced maturation of dendritic cells and activation of T cells and NK cells when administered to a subject suffering from cancer.

### BRIEF DESCRIPTION OF THE DRAWINGS

The embodiments, together with further objects and advantages thereof, may best be understood by making reference to the following description taken together with the accompanying drawings, in which:

FIG. 1 schematically illustrates adenovirus constructs used in the Examples, showing the gene arrangements and locations.

FIG. 2 illustrates relative cell viability of Panc01 and MiaPaCa-2 cells after infection with various recombinant adenoviruses at different multiple of infections (MOls). Cell viability was measured 4 days post infection (d.p.i) and is presented as percentage relative to non-infected control cells.

FIG. 3 illustrates replication of recombinant viruses in Panc01 and MiaPaCa-2 cells. Cells were transduced with different viruses at MOI=50. Viral genomic DNA was isolated at days 0, 1, 2, and 3 after transduction and quantified using real-time PCR. Values show the virus genome copy numbers calculated based on standard curve. The data are shown as means+standard deviation (SD) with triplicate samples.

FIG. 4 illustrates the expression level of transgenes TNFSF9 or TNFSF18 at 2 days after transduction of Panc01 and MiaPaCa-2 cells. Expression levels were reported as mean fluorescent intensity (MFI)

FIG. 5 illustrates cell surface exposure of calreticulin (CRT) and the release of ATP at 2 days after virus transduction. CRT was measured by flow cytometry and shown as MFI and ATP was measured using ATP determination kit (Invitrogen) and presented as artificial units (a.u.).

FIG. 6 illustrates expression levels of human dendritic cell (DC) maturation markers (CD80, CD86, CD40 and CCR7) analyzed by flow cytometry and presented as MFI, after 18 hours of co-culturing immature DC with virus-transduced cells.

FIG. 7A illustrates expression levels of tumor infiltrating CD8+ T cells, CD4+ T cells, and CD56+NK cells maturation markers (CD69 and CD107a) analyzed by flow cytometry and presented as MFI, at 3 days after intra-tumoral treatment with different viruses.

FIG. 7B illustrates IFN-γ release from splenocytes harvested from different virus-treated mice, and re-stimulated with Panc02 cells.

FIG. 8 illustrates subcutaneous Panc02 tumor growth and mice survival after treatment with different adenoviruses.

FIG. 9 illustrates subcutaneous Panc01 tumor (human xenograft) growth on immunodeficient mice and the mice survival after treatment.

FIG. 10 illustrates subcutaneous NSX2 tumor growth on mice and the mice survival after treatment.

### DETAILED DESCRIPTION

The present invention generally relates to adenoviruses, and in particular to recombinant adenoviruses useful in treatment of cancers.

The recombinant adenoviruses of the embodiments have enhanced therapeutic effect in terms of significantly inhibiting tumor growth and significantly prolonging survival in subjects following tumor implantation. This enhanced therapeutic effect is achieved by engineering the adenovirus to comprise a nucleic acid sequencing encoding a Helicobacter pylori neutrophil-activating protein (NAP) and/or a nucleic acid sequence encoding an immunologically equivalent fragment of NAP in combination with a nucleic acid sequence encoding an immunomodulator capable of inducing an immune response in a subject when the adenovirus is administered to the subject. The adenovirus is, thus, a recombinant or engineered virus comprising nucleic acid sequences encoding NAP, and/or the immunologically equivalent fragment of thereof, and the immunomodulator.

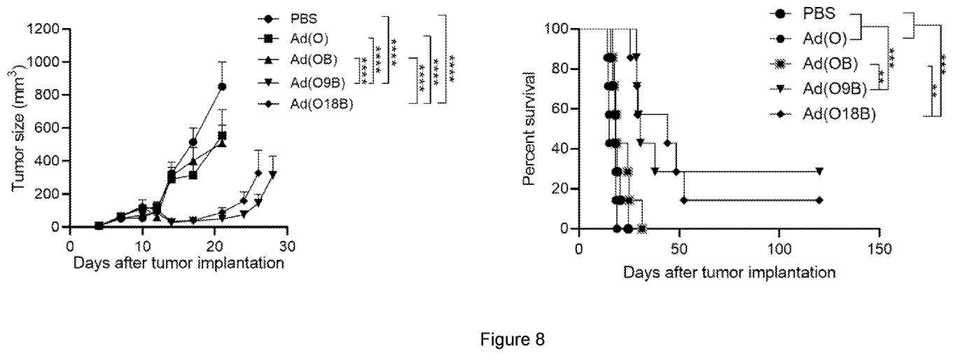

Adenoviruses comprising a nucleic acid sequence encoding NAP are known in the art as mentioned in the background section. However, experimental data as presented herein shows that such adenoviruses comprising a nucleic acid sequence encoding NAP but lacking any nucleic acid sequence encoding an immunomodulator could marginally inhibit tumor growth and did not result in any significant prolonged survival of subject following tumor implantation (FIG. 8).

However, combining NAP, or an immunologically fragment thereof, with another immunomodulator significantly improved the therapeutic effect of the adenovirus (FIG. 8). This was highly surprising since co-expression of NAP and another heterologous gene in viruses has been shown to lead to an enhanced antibody response against the gene product of the heterologous gene when administered to a subject and where such an antibody response thereby will hamper or inhibit the function of the gene product (lankov et al., Measles virus expressed Helicobacter pylori neutrophil-activating protein significantly enhances the immunogenicity of poor immunogens, Vaccine (2013) 31(42): 4795-4801). Hence, one would expect that co-expressing NAP, and/or an immunologically active fragment thereof, together with an immunomodulator in adenoviruses would, when administered to a subject, trigger an antibody response in the subject against the immunomodulator. The antibody response raised against the immunomodulator would then be expected to inhibit and block the action of the immunomodulator in the subject, i.e., would prevent or at least significantly inhibit induction of an immune response in the subject as otherwise induced by the immunomodulator.

Experimental data as presented herein, however, show that co-expressing NAP and the immunomodulator in adenoviruses did not negatively affect the immune response inducing function of the immunomodulator. In clear contrast, in addition to the enhanced therapeutic effect, co-expression of NAP, and/or the immunologically active fragment thereof, and the immunomodulator induced immunogenic cell death (ICD) of cancer cells.

ICD, also referred to as immunogenic apoptosis, is a form of cell death resulting in a regulated activation of the immune response. This cell death is characterized by apoptotic morphology, maintaining membrane integrity. Immunogenic death of cancer cells induces an effective antitumor immune response through activation of dendritic cells (DCs) and consequent activation of specific T cell response. Hence, the induction of ICD by the adenovirus of the invention means that the adenovirus is highly effective in antitumor therapy.

Furthermore, the adenovirus of the present invention co-expressing NAP, and/or the immunologically active fragment thereof, and the immunomodulator induced DC maturation and activation of T cells, including CD4+ T cells and CD8+ T cells, and natural killer (NK) cells, including CD56+NK cells. Furthermore, treatment with the adenovirus of the invention led to generation of immunological memory as indicated by endogenous splenocytes from treated subjects also reacted with tumor cells by release of significant amounts of interferon gamma (IFN-γ).

The co-expression of NAP, and/or the immunologically active fragment thereof, and the immunomodulator in the adenovirus of the invention did not negatively affect the expression of the immunomodulator in transduced cancer cells. In addition, the adenovirus could efficiently replicate in the cancer cells and specifically kill the cancer cells.

Unless defined otherwise, all technical and scientific terms used herein have the meaning commonly understood by a person skilled in the art to which the present invention belongs. The following references (Singleton et al., Dictionary of microbiology and molecular biology. 3rd ed, Revised, 2007, ISBN: 9780470035450; Walker, The Cambridge dictionary of science and technology. 1990, ISBN: 9780521394413; Rieger et al., Glossary of Genetics: Classical and Molecular. 5th ed., 1991, ISBN: 9783540520542; Hale, HarperCollins dictionary of biology. 1991, ISBN: 9780064610155; Lewin, Gene XII. 2017, ISBN: 978-1-2841-0449-3; Knipe et al., Field's Virology. 6th ed., 2013, ISBN: 978-1-4511-0563-6) provide a general definition of many of the terms used in this invention. For clarity, the following definition is used herein.

The terms “nucleic acid sequence” or “nucleotide sequence” refer to a polymer composed of nucleotides, such as ribonucleotides, deoxyribonucleotides, related naturally occurring structural variants, and/or synthetic non-naturally occurring analogs thereof, linked via phosphodiester bonds, related naturally occurring structural variants, and/or synthetic non-naturally occurring analogs thereof. Examples of such nucleic acid or nucleotide sequences are deoxyribonucleic acid (DNA) sequences and ribonucleic acid (RNA) sequences. In a particular embodiment, the nucleic acid or nucleotide sequences are DNA sequences.

An aspect of the invention relates to an adenovirus comprising a nucleic acid sequence encoding a Helicobacter pylori neutrophil-activating protein (NAP) and/or a nucleic acid sequence encoding an immunologically equivalent fragment of NAP. The adenovirus also comprises a nucleic acid sequence encoding an immunomodulator capable of inducing an immune response in a subject.

The adenovirus is thereby capable of co-expressing NAP, and/or the immunologically equivalent fragment of thereof, in addition to the immunomodulator. Hence, the immunomodulator is different from NAP or the immunologically active fragment of NAP.

The immunomodulator is capable of inducing an immune response in a subject when the adenovirus is administered to the subject. In an embodiment, the immunomodulator is capable of inducing DC maturation. Cancer cells transduced with the adenovirus of the invention were able to mature and activate DCs when co-cultured as indicated by elevated surface expression of maturation and activation markers including cluster of differentiation 80 (CD80), also referred to as B7-1, CD40, CD86, also referred to as B7-2, and C-C chemokine receptor type 7 (CCR7)

In another embodiment, the immunomodulator is capable of inducing T cell activation, in particular CD4+ T cell activation, CD8+ T cell activation, or both CD4+ T cell and CD8+ T cell activation. Subjects implanted with cancer cells and treated with adenovirus of the present invention had an enhanced activation of tumor infiltrating CD4+ and CD8+ T cells. The adenovirus treatment led to an activation of these CD4+ and CD8+ T cells as indicated by upregulation of the surface markers CD69 and CD107a, also referred to as lysosomal-associated membrane protein 1 (LAMP-1) or lysosome-associated membrane protein 1.

In a further embodiment, the immunomodulator is capable of inducing NK cell activation, in particular CD56+NK cell activation. Subjects implanted with cancer cells and treated with adenovirus of the present invention had an enhanced activation of tumor infiltrating NK cells. The adenovirus treatment lead to an activation of these NK cells as indicated by upregulation of the surface markers CD69 and CD107a.

In preferred embodiments, the immunomodulator is capable of inducing DC maturation and T cell activation; inducing DC maturation and NK cell activation or inducing T cell activation and NK cell activation in the subject. In a currently preferred embodiment, the immunomodulator is capable of inducing DC maturation, T cell activation and NK cell activation in the subject.

In an embodiment, the immunomodulator is a tumor necrosis factor (TNF) superfamily (TNFSF) member.

TNFSF is a protein superfamily of type II transmembrane proteins containing TNF homology domain and forming trimers. Members of TNFSF can be released from the cell membrane by extracellular proteolytic cleavage and function as a cytokine. These proteins are expressed predominantly by immune cells and they regulate diverse cell functions, including immune response and inflammation, but also proliferation, differentiation, apoptosis and embryogenesis.

In an embodiment, the TNFSF member is selected from the group consisting of TNFSF1, TNFSF2, TNFSF4, TNFSF5, TNFSF7, TNFSF9, TNFSF14, TNFSF18, and a combination thereof.

TNFSF1, also referred to as lymphotoxin-alpha (LT-a) or TNF-beta (TNF-β), exhibits anti-proliferative activity and causes the cellular destruction of tumor cells. TNFSF1 is involved in induction of inflammation and antiviral response, development of secondary lymphoid organs, and regulation of cell survival, proliferation, differentiation and apoptosis.

TNFSF2, also referred to as tumor necrosis factor (TNF), TNF-α, cachexin or cachectin, has a role in regulation of immune cells, induction of fever, cachexia, inflammation and apoptosis.

TNFSF2 also inhibits tumorigenesis.

TNFSF4, also referred to as OX40 ligand, CD252, Gp34 or CD134L, induces activation of T cell immune response by T cell co-stimulation.

TNFSF5, also referred to as CD40 ligand (CD40L), regulates the adaptive immune response by activating antigen presenting cells (APCs).

TNFSF7, also referred to as CD27 ligand (CD27L) or CD70, regulates B cell activation and T cell homeostasis.

TNFSF9, also referred to as CD137 ligand or 4-1BB ligand (4-1BBL), is found on APCs and binds to CD137, also referred to as 4-1BB, expressed on activated T cells.

TNFSF14, also referred to as LIGHT, CD258 or HVEML, regulates B cell activation and T cell homeostasis.

TNFS18, also referred to as glucocorticoid-induced tumor necrosis factor receptor-related protein (GITR) ligand (GITRL), activation-induced TNFR member ligand (AITRL) or TL-6, is a cytokine that is ligand for receptor TNF receptor superfamily 18 (TNFRSF18), also referred to as GITR or AITR. TNFS18 modulated T cell survival and is thought to be of importance in the interaction between T cells and endothelial cells.

In an embodiment, the immunomodulator is a membrane bound immunomodulator. In such a case, the immunomodulator expressed in cancer cells infected by the adenovirus according to the present invention is preferably bound to the cell membrane of the infected cancer cells. The membrane bound immunomodulator is then capable of inducing a local immune response in the subject at the site of viral infection of cancer cells, i.e., at the site of the cancer cells or tumor. Such membrane bound immunomodulators are generally preferred over soluble immunomodulators, which may be transported away from the site of cancer cells and thereby be less effective in inducing an immune response at the desired site in the subject.

In an embodiment, the adenovirus comprises a nucleotide sequence encoding one TNFSF member, preferably selected from the group presented above. In such a case, the adenovirus expresses a single TNFSF protein. In another embodiment, the adenovirus comprises a nucleotide sequence encoding multiple, i.e., at least two, different TNFSF members, or multiple nucleotide sequences encoding respective different TNFSF members.

In a preferred embodiment, the TNFSF member is selected from the group consisting of TNFSF5, TNFSF9, TNFSF14, TNFSF18, and a combination thereof.

In a more preferred embodiment, the TNFSF member is selected from the group consisting of TNFSF9, TNFSF18, and a combination thereof.

In an embodiment, the adenovirus comprises a nucleic acid sequence encoding TNFSF9. In another embodiment, the adenovirus comprises a nucleic acid sequence encoding TNFSF18. In a further embodiment, the adenovirus comprises a nucleic acid sequence encoding TNFSF9 and a nucleic acid sequence encoding TNFSF18 or a nucleic acid sequence encoding TNFSF9 and TNFSF18.

Helicobacter pylori neutrophil-activating protein, or NAP or HP-NAP for short, is a dodecameric protein that acts as a virulence factor in H. pylori bacterial infection. It is made of 12 monomeric subunits and each subunit is comprised of four a-helices. The surface of NAP is highly positively charged and has capacity of interacting with and activating human white blood cells (WBCs), also denoted leukocytes.

NAP plays a critical role in migration of neutrophils to inflamed tissue during H. pylori infection. NAP promotes strong binding of neutrophils and monocytes binding to endothelium and extravasation by upregulating surface expression of B2 integrin. It can also active neutrophils in producing reactive oxygen species (ROS) and myeloperoxidases. NAP also activates secretion of other pro-inflammatory cytokines, such as TNF-α and interleukin 8 (IL-8), also referred to as chemokine (C-X-C motif) ligand 8 (CXCL8), which in turn induce adhesion molecules expression like vascular cell adhesion molecule (V-CAM), intercellular adhesion molecule (I-CAM) and secretion of IL-8 by endothelia cells. In addition, NAP can also induce neutrophil secretion of several cytokines and chemokines expression, such IL-8, macrophage inflammatory protein 1 alpha (MIP-1c) and MIP-1B, also referred to as chemokine (C-C motif) ligand 4 (CCL4). These cytokines and chemokines in turn attract, by chemotaxis, neutrophils to the site of inflammation.

NAP is a toll-like receptor 2 (TLR-2) agonist, is chemotactic for neutrophils, monocytes and can mature DCs both in vitro and in vivo. It can also stimulate secretion of IL-12 and IL-23, which are Th-1 polarizing cytokines. NAP stimulates monocytes to differentiate and mature into DCs by upregulating expression of HLA—antigen D related (HLA-DR), CD80 and CD86. It also has pivotal immunoregulatory functions in aiding cytotoxic T cells and NK cells activation. NAP can induce T cells to secrete high level of IFN-γ and low level of IL-4, also suggesting a Th1 polarizing response. This is coincident with report that H. pylori infected humans indicate a strong Th1 polarizing response.

In an embodiment, NAP preferably comprises, or consists of, an amino acid sequence selected from any of the following sequences:

(SEQ ID NO: 3)
MKTFEILKHLQADAIVLFMKVHNFHWNVKGTDFFNVHKATEEIYEEFADM
FDDLAERIVQLGHHPLVTLSEAIKLTRVKEETKTSFHSKDIFKEILEDYK
YLEKEFKELSNTAEKEGDKVTVTYADDQLAKLQKSIWMLQAHLA
(SEQ ID NO: 4)
MKTFEILKHLQADAIVLFMKVHNFHWNVKGTDFFNVHKATEEIYEEFADM
FDDLAERIVQLGHHPLVTLSEAIKLTRVKEETKTSFHSKDIFKEILEDYK
HLEKEFKELSNTAEKEGDKVTVTYADDQLAKLQKSIWMLQAHLA
(SEQ ID NO: 5)
MKTFEILKHLQADAIVLFMKVHNFHWNVKGTDFFNVHKATEEIYEEFADM
FDDLAERIVQLGHHPLVTLSEALKLTRVKEETKTSFHSKDIFKEILEDYK
YLEKEFKELSNTAEKEGDKVTVTYADDQLAKLQKSIWMLQAHLA
(SEQ ID NO: 6)
MKTFEILKHLQADAIVLFMKVHNFHWNVKGTDFFNVHKATEEIYEEFADM
FDDLAERIVQLGHHPLVTLSEALKLTRVKEETKTSFHSKDIFKEILEDYK
HLEKEFKELSNTAEKEGDKVTVTYADDQLAKLQKSIWMLQAHLA
(SEQ ID NO: 7)
MKTFEILKHLQADAIVLFMKVHNFHWNVKGTDFFNVHKATEEIYEGFADM
FDDLAERIVQLGHHPLVTLSEAIKLTRVKEETKTSFHSKDIFKEILEDYK
YLEKEFKELSNTAEKEGDKVTVTYADDQLAKLQKSIWMLQAHLA
(SEQ ID NO: 8)
MKTFEILKHLQADAIVLFMKVHNFHWNVKGTDFFNVHKATEEIYEGFADM
FDDLAERIVQLGHHPLVTLSEALKLTRVKEETKTSFHSKDIFKEILEDYK
YLEKEFKELSNTAEKEGDKVTVTYADDQLAKLQKSIWMLQAHLA
(SEQ ID NO: 9)
MKTFEILKHLQADAIVLFMKVHNFHWNVKGTDFFNVHKATEEIYEGFADM
FDDLAERIVQLGHHPLVTLSEAIKLTRVKEETKTSFHSKDIFKEILEDYK
HLEKEFKELSNTAEKEGDKVTVTYADDQLAKLQKSIWMLQAHLA
(SEQ ID NO: 10)
MKTFEILKHLQADAIVLFMKVHNFHWNVKGTDFFNVHKATEEIYEGFADM
FDDLAERIVQLGHHPLVTLSEALKLTRVKEETKTSFHSKDIFKEILEDYK
HLEKEFKELSNTAEKEGDKVTVTYADDQLAKLQKSIWMLQAHLA

An immunological equivalent fragment of NAP is a fragment including at least one polypeptide domain of at least 20 amino acid residues, such as at least 20 consecutive amino acid residues, preferably at least 30 amino acid residues, such as at least 30 consecutive amino acid residues, and more preferably at least 40 amino acid residues, such as at least 40 consecutive amino acid residues, of NAP.

Non-limiting but illustrative examples of immunologically equivalent fragments of NAP include:

(SEQ ID NO: 11)
EILKHLQADAIVLFMKVHNFHWNVKGTDFFNVHKAT
corresponding to amino acids no. 5-40
of SEQ ID NO: 3-10
(SEQ ID NO: 12)
NTAEKEGDKVTVTYADDQLAKLQKSIWMLQAHLA
corresponding to amino acids no. 110-144
of SEQ ID NO: 3-10
(SEQ ID NO: 13)
ATEEIYEEFADMFDDLAERIVQLGHHPLVTLSEALK
corresponding to amino acids no. 39-74
of SEQ ID NO: 5-6
(SEQ ID NO: 14)
LTRVKEETKTSFHSKDIFKEILEDYKHLEKEFKELS
corresponding to amino acids no. 75-110
of SEQ ID NO: 5, 10

More information of immunologically equivalent fragments of NAP can be found in EP 1 767 214 B1, the teaching of which with regard to immunological equivalent fragments of HP-NAP disclosed in paragraph 32, “Definition of the dominant T-cell epitopes of HP-NAP recognized by HP-NAP-specific T-cells derived from the gastric infiltrates induced by H. pylori” is hereby incorporated by reference.

In an embodiment, the adenovirus comprises a nucleic acid sequence encoding a single NAP, nucleic acid sequences encoding multiple different NAPs, a nucleic acid sequence encoding a single immunologically active fragment of NAP, nucleic acid sequences encoding multiple different immunologically active fragments of NAP, or at least one nucleic acid sequence encoding at least one NAP and at least one nucleic acid sequence encoding at least one immunologically active fragment of NAP.

In an embodiment, the adenovirus further comprises a nucleic acid sequence encoding a self-cleaving peptide positioned between the nucleic acid sequence encoding NAP, and/or the immunologically active fragment of NAP, and the nucleic acid sequence encoding the immunomodulator.

A self-cleaving peptide as referred to herein is a peptide that can induce ribosomal skipping during translation of a protein in a cell. The apparent cleavage is triggered by ribosomal skipping of the peptide bond between proline (P) and glycine (G) in the C-terminal of the self-cleaving peptide, resulting in a peptide or protein located upstream of the self-cleaving peptide to have extra amino acids on its C-terminal end while a peptide or protein located downstream the self-cleaving peptide will have an extra proline on its N-terminal end.

In an embodiment, the self-cleaving peptide is a self-cleaving 2A peptide, also referred to as a 2A peptide. Such a self-cleaving 2A peptide comprises a core sequence motif of DxExNPGP (SEQ ID NO: 15). In an embodiment, the self-cleaving 2A peptide is selected from the group consisting of Thosea asigna virus 2A peptide (T2A), Porcine teschovirus-1 2A peptide (P2A), Equine rhinitis A virus 2A peptide (E2A) and foot-and-mouth disease virus 2A (F2A). In an embodiment, T2A consists of the amino acid sequence EGRGSLLTCGDVEENPGP (SEQ ID NO: 16) or GSGEGRGSLLTCGDVEENPGP (SEQ ID NO: 17). In an embodiment, P2A consists of the amino acid sequence ATNFSLLKQAGDVEENPGP (SEQ ID NO: 18) or GSGATNFSLLKQAGDVEENPGP (SEQ ID NO: 19). In an embodiment, E2A consists of the amino acid sequence QCTNYALLKLAGDVESNPGP (SEQ ID NO: 20) or GSGQCTNYALLKLAGDVESNPGP (SEQ ID NO: 21). In an embodiment, F2A consists of the amino acid sequence VKQTLNFDLLKLAGDVESNPGP (SEQ ID NO: 22) or GSGVKQTLNFDLLKLAGDVESNPGP (SEQ ID NO: 23).

Inclusion of a nucleic acid sequence encoding the self-cleaving peptide in between the nucleic acid sequences encoding NAP, and/or the immunologically fragment thereof, and the immunomodulator facilitates correct translation and folding of NAP, and/or the immunologically fragment thereof, and the immunomodulator independently of each other and any other proteins in the genome of the adenovirus.

In an embodiment, the adenovirus is an oncolytic adenovirus. An oncolytic adenovirus is an adenovirus that can selectively replicate in cancer cells. Such an oncolytic adenovirus has preferential replication in cancer cells, i.e., oncotropism, and is preferably capable of lysis of the cancer cells, i.e., oncolysis.

In an embodiment, the adenovirus is engineered to be oncolytic, i.e., to selectively replicate in cancer cells. In a preferred embodiment, the oncolytic adenovirus comprises a mutated adenovirus early region 1A (E1A) gene encoding a mutated E1A protein having a lower Rb binding capability as compared to a wild-type E1A protein.

The E1A protein of adenoviruses binds to the cellular Rb protein of the infected host cell. The binding of E1A protein to cellular Rb protein releases E2F, which activates transcription of other viral genes, such as E2 (encoding proteins involved in virus replication), E3 (encoding proteins inhibiting the antiviral immune response of the host), and E4 (encoding proteins involved in viral ribonucleic acid (RNA) transport), and of cellular genes that activate the cell cycle.

Lowering, such as inhibiting or fully preventing, the E1A protein from binding to the cellular Rb protein makes the adenovirus cancer or tumor cell selective. Thus, an adenovirus comprising a mutated E1A gene encoding the mutated E1A protein can replicate in and lyse cancer cells but not in healthy or normal, i.e., non-cancer, cells.

In an embodiment, the mutated E1A gene comprises a 24 base pair (bp) deletion of nucleotides 919 to 943 of wild-type E1A gene. This 24 bp deletion corresponds to nucleotides cttacctgccaggaggctggcttt (SEQ ID NO: 24). These 24 nucleotides encode amino acids 121 to 128 of the E1A protein. The mutated E1A protein thereby lacks amino acids 121 to 128 of wild-type E1A protein corresponding to LTCHEACF (SEQ ID NO: 25).

In an embodiment, the adenovirus lacks the nucleic acid sequence 19-kDa adenovirus E1B protein and the nucleic acid sequence encoding 55-kDa adenovirus E1B protein.

In an embodiment, one of the nucleic acid sequences encoding 19-kDa adenovirus E1B protein and 55-kDa adenovirus E1B protein is replaced by the nucleic acid sequence encoding NAP and/or the nucleic acid sequence encoding the immunologically equivalent fragment of NAP and the other of the nucleic acid sequences encoding 19-kDa adenovirus E1B protein and 55-kDa adenovirus E1B protein is replaced by the nucleic acid sequence encoding the immunomodulator.

Hence, in a preferred embodiment, the nucleic acid sequences of the adenovirus encoding the 19-kDa adenovirus E1B protein and the 55-kDa adenovirus E1B protein are replaced by the nucleic acid sequences encoding NAP, and/or the immunologically equivalent fragment thereof, and the immunomodulator.

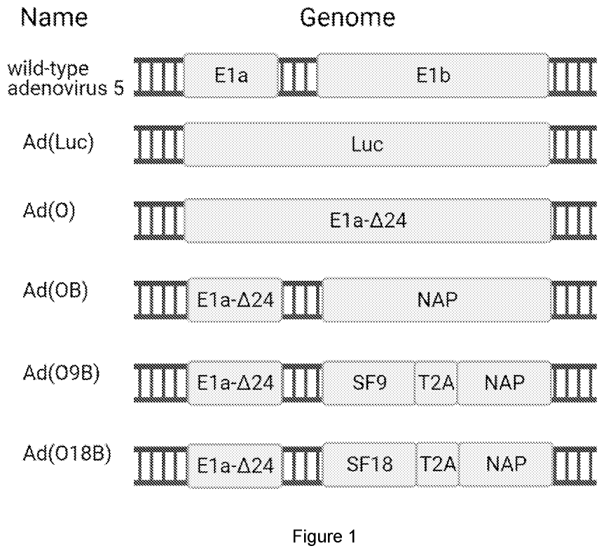

In an embodiment, the adenovirus comprises the nucleic acid sequence encoding the immunomodulator followed by the nucleic acid sequence encoding the self-cleaving peptide and followed by the nucleic acid sequence encoding NAP, and/or the immunologically active fragment thereof as shown in FIG. 1. In another embodiment, the adenovirus comprises the nucleic acid sequence encoding NAP, and/or the immunologically active fragment thereof, followed by the nucleic acid sequence encoding the self-cleaving peptide and followed by the nucleic acid sequence encoding the immunomodulator.

In an embodiment, the adenovirus is a human adenovirus type 5, preferably an oncolytic human adenovirus type 5.

Human adenovirus type 5 (Ad5), belongs to group C, is a virus formed by a protein icosahedral capsid that packages a linear deoxyribonucleic acid (DNA) of 36 kilobases. In adult humans, infection with Ad5 is usually asymptomatic and in children it causes a common cold and conjunctivitis. In general, Ad5 infects epithelial cells, which in the course of a natural infection are the cells of the bronchial epithelium. It enters the cell by means of the interaction of a fiber, a viral protein that extends as an antenna from the twelve vertices of the capsid, with the cellular protein Coxsackie-adenovirus receptor (CAR) involved in intercellular adhesion. When the viral DNA arrives within the cell nucleus, it begins an ordered transcription of the early genes (E1 to E4) of the virus. The first viral genes that are expressed are the genes of the early region 1A (E1A). E1A binds to the cellular protein Rb to release E2F, which activates the transcription of other viral genes, such as E2 (encoding proteins involved in virus replication), E3 (encoding proteins inhibiting the antiviral immune response of the host), and E4 (encoding proteins involved in viral ribonucleic acid (RNA) transport), and of cellular genes that activate the cell cycle. Furthermore, E1B binds to p53 to activate the cell cycle and to prevent apoptosis of the infected cell. The expression of the early genes leads to replication of the virus DNA, and once the DNA has replicated, the major late promoter is activated and drives transcription of messenger RNA (mRNA) that upon differential splicing generates all the RNAs encoding for the structural proteins that form the capsid.

Another aspect of the invention relates to an adenovirus according to the embodiments for use as a medicament.

A further aspect of the invention relates to an adenovirus according to the embodiments for use in treatment of cancer.

A related aspect of the invention defines use of an adenovirus according to the embodiments for the manufacture of a medicament for treatment of cancer.

The present invention further relates to a method for treatment of cancer. The method comprises administering an effective amount of an adenovirus according to the embodiment to a subject suffering from cancer.

“Treating” or “treatment” as used herein and is well understood in the art, means an approach for obtaining beneficial or desired results, including clinical results. Beneficial or desired clinical results could include, for instance, alleviation or amelioration of one or more symptoms or conditions, diminishment of extent of cancer disease, stabilized state of cancer disease, i.e., prevent worsening, preventing spread of cancer disease, delay or slowing of disease progression, amelioration or palliation of the cancer disease state, diminishment of the reoccurrence of cancer disease, and remission. “Treating” or “treatment” may also prolong survival as compared to expected survival if not receiving any treatment. Treatment of cancer as used herein also encompasses inhibiting cancer in a subject and prophylactic treatment.

“Preventing” or “prophylaxis” as used herein and is well understood in the art, means an approach in which a risk of developing a cancer disease or condition is reduced or prevented, including prolonging or delaying cancer disease development. For instance, a patient predisposed to develop a cancer disease, such as due to genetic or hereditary predisposition, could benefit for administration of the adenovirus of the embodiments to prevent, reduce the risk of, delaying and/or slowing development of the cancer disease.

The cancer or cancer disease is preferably selected from the group consisting of carcinoma, such as pancreatic cancer, breast cancer, lung cancer, liver cancer, or kidney cancer; sarcoma, such as osteosarcoma or liposarcoma; lymphoma, such as non-Hodgkin lymphoma or Hodgkin lymphoma; leukemia, such as acute leukemia or chronic leukemia; seminoma; germinoma; dysgerminoma; and blastoma, such as glioblastoma or neuroblastoma. In a particular embodiment, the cancer is carcinoma, such as pancreatic cancer.

The patient is preferably a human patient. The embodiments may, however, also be applied in veterinary applications, i.e. non-human patients, such as non-human mammals including, for instance, primates, monkeys, apes, cattle, sheep, pigs, goats, horses, cats, dogs, mice, rats and guinea pigs.

The adenovirus may be administered to the patient according to various routes including, for instance, intravenous, subcutaneous, intraperitoneal, intramuscular or intratumoral administration.

The adenovirus is typically administered in the form of a pharmaceutical composition comprising the adenovirus. The pharmaceutical composition may additionally comprise one or more pharmaceutically acceptable carriers, vehicles and/or excipients. Non-limiting examples of such pharmaceutically acceptable carriers, vehicles and excipients include injection solutions, such as saline or buffered injection solutions.

The pharmaceutical composition preferable comprises an effective amount of adenoviruses. As used herein, “effective amount” indicates an amount effective, at dosages and for periods of time necessary to achieve a desired result. For example, in the context of inhibiting a tumor growth an effective amount is an amount that, for example, induces remission, reduces tumor burden, and/or prevents tumor spread or growth compared to the response obtained without administration of the cells. Effective amounts may vary according to factors, such as the disease state, age, sex, weight of the patient.

### EXAMPLES

### Example 1 Construction of Adenoviruses

### Material and Methods

### Adenoviral Plasmid and Viruses

The recombinant adenoviral genome was engineered using pAdEasy system. A DNA construct was synthesized containing corresponding sequences as wild-type adenovirus genome, wherein the E1A coding sequence was mutated with a 24 bp deletion (E1a-A24) (Fueyo, J., et al., A mutant oncolytic adenovirus targeting the Rb pathway produces anti-glioma effect in vivo, Oncogene (2000) 19(1): 2-12), the native p19K and p55K coding sequence were replaced by coding sequence of TNFSF9 (SF9) or TNFSF18 (SF18), self-cleavage 2A peptide from Thosea asigna virus 2A (T2A), and neutrophil-activating protein (NAP) from Helicobacter pylori. Both human and murine TNFSF9 or TNFSF18 were constructed and were used to match the experiment condition (i.e., human genes were used in human cell line and murine genes were used in murine models). These constructs were cloned into empty pShuttle to generate pSh(09B) and pSh(O18B). The pShuttle plasmids were further recombined with pAdEasyf35, to generate pAd(09B) and pAd(O18B), which were used for the production of the oncolytic viruses Ad(O9B) and Ad(O18B). The non-replicating adenovirus Ad(Luc) was produced in a similar way, and was used as a negative control. Additionally, two control viruses were produced in a similar way, wherein Ad(O) had E1A mutated with 24 bp deletion, but had no transgene expression and Ad(OB) had E1A mutated with 24 bp deletion and had only NAP as transgene expression.

### Cell Lines and Culture Conditions

Human embryonic retinoblastoma 911 cells (Crucell, Leiden, The Netherlands) were cultured in a humidified incubator (5% CO₂, 37° C.). The cell line was maintained in Dulbecco's Modified Eagle's medium (DMEM) Glutamax supplemented with 10% heat-inactivated fetal bovine serum (FBS), 1 mM sodium pyruvate and 100 U/ml penicillin-streptomycin (PEST). All of the used materials were purchased from Thermo Fisher Scientific.

### Virus Production and Amplification

The recombinant adenoviruses were produced after transfection of 90% confluent 911 cells with 12 μg Pacl-digested pAd5(E1AD24-A-B) DNA and the addition of polyethylenimine (PEI, Polysciences, Inc.). Cytopathic effects (CPE) were evident within 5 days as almost half of the cells had a rounded nucleus and were detached. The transfected cells were collected on day 6 and lysed by repeated freezing and thawing cycles to release the cytoplasmic viral particles. The adenoviral titer was increased by successive transduction rounds of 911 cells. The viruses were purified by CsCl gradient ultracentrifugation at 25,000 rounds per minute (rpm) at 4° C. for 2 hours and dialyzed in storage (10 mM Tris-HCl (pH 8.0), 2 mM MgCl₂ and 4% w/v sucrose). The purified viruses were aliquoted and stored at −80° C.

### Results

We are able to construct all virus DNA and produce the viruses in high titer. The virus constructs, their names and genome arrangements are presented in FIG. 1. Transgene(s) (Luc, SF9, SF18, NAP) and mutations (E1a-A24) are indicated and the relevant location of each modifications in the genome according to wild type genome arrangement are also indicated.

### Example 2 Oncolytic Virus Co-Expressing TNFSF9 or TNFSF18 Together with NAP Specifically Killed Cancer Cells and Replicated Efficiently

### Cell Lines and Culture Conditions

Human pancreatic cancer cell lines Panc01 (purchased from ATCC, USA), and MiaPaCa-2 (purchased from ATCC, USA) were cultured in a humidified incubator (5% CO₂, 37)° ° C. The cell lines were maintained in Dulbecco's Modified Eagle's medium (DMEM) Glutamax supplemented with 10% heat-inactivated fetal bovine serum (FBS), 1 mM sodium pyruvate and 100 U/ml penicillin-streptomycin (PEST). All of the used materials were purchased from Thermo Fisher Scientific.

### MTS Killing Assay

Panc01 and MiaPaCa-2 cells (1×10⁴ cells) were transduced (mixing cells and virus at corresponding number/volume in 200 UL culture media) with the different viruses at multiple of infection (MOI) of 0.1-1000 and plated in a 96-well plate, and cell viability was measured 5 days later with Alamar Blue compound. Cell viability was determined as percentage of live cells compared to the untreated control cells. Results represent the mean of three independent experiments.

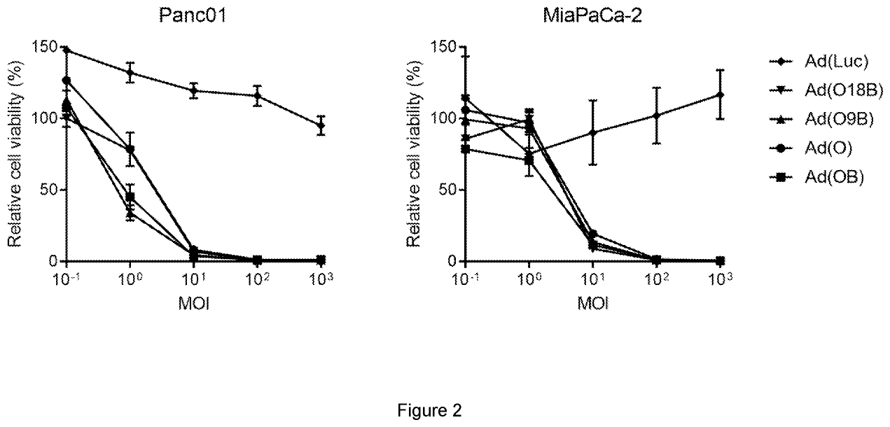

The control virus Ad(Luc) did not show any cell killing, while both engineered Ad(O9B) and Ad(O18B) showed similar cell killing ability as compared to Ad(O) and Ad(OB), indicating that insertion of additional transgenes, i.e., TNFSF9 or TNFSF18, did not negatively affect virus killing ability (FIG. 2).

### Replication Assay

Panc01 and MiaPaCa-2 cells (1×10⁴ cells) were transduced with the different viruses at MOI=50 and plated in a 96-well plate. Viral DNA was extracted from the cells at different timepoints using the High Pure Viral Nucleic Acid kit (Roche) and quantitative polymerase chain reaction (qPCR) was performed using adenovirus specific primers (Forward primer: CATCAGGTTGATTCACATCGG (SEQ ID NO: 1) Reverse primer:

(SEQ ID NO: 2))
GAAGCGCTGTATGTTGTTCTG.

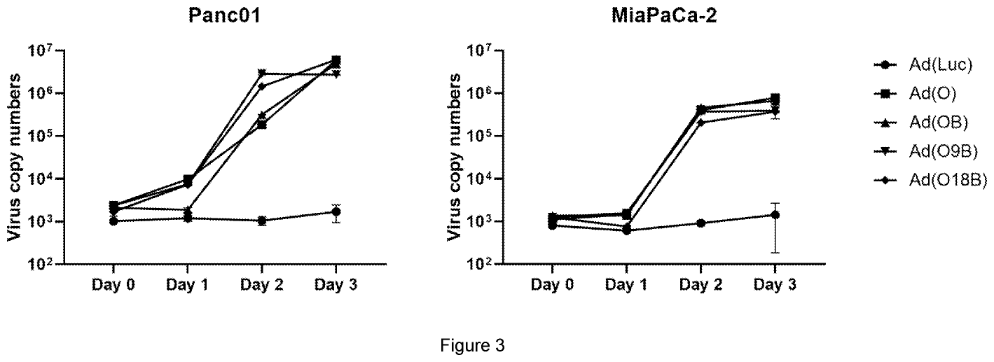

The control virus Ad(Luc) did not replicate in either of the cell lines, while recombinant viruses Ad(O9B) and Ad(O18B) replicated in both of the cell lines (FIG. 3). In Panc01 cel line, the insertion of either transgene (TNFSF9 or TNFSF18) actually seemed to increase the viral replication as compared to the viruses Ad(O) and Ad(OB). The replication was similar for all oncolytic viruses in MiaPaCa-2 cell line.

### Example 3 Co-Expression of TNFSF9 or TNFSF18 Together with NAP Did not Negatively Affect Transgene Expression

### Transgene Expression

Panc01 and MiaPaCa-2 cells (1×10⁶ cells) were transduced with various viruses at MOI=50 and plated in a 6-well plate. Transgene expression of TNFSF9 or TNFSF18 was determined with flow cytometry at day 2 after transduction.

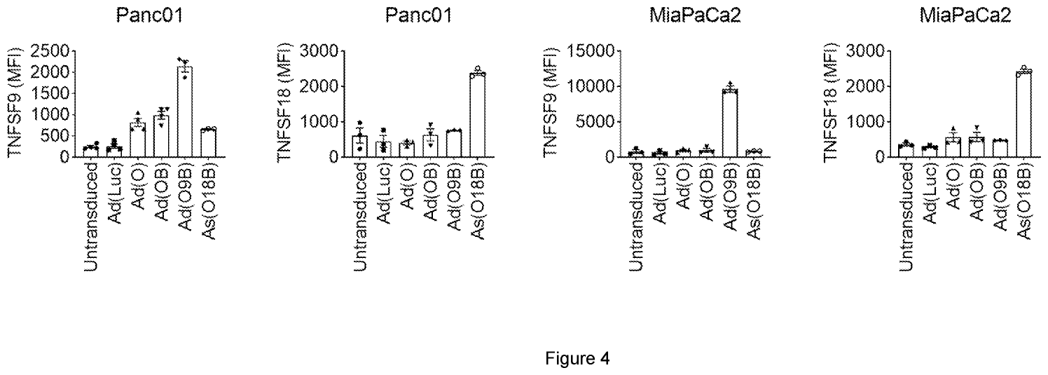

As expected, none of the cells transduced with control viruses expressed either of the transgenes TNFSF9 or TNFSF18. However, cells transduced with Ad(O9B) virus expressed high level of TNFSF9 and cells transduced with Ad(O18B) virus expressed high level of TNFSF18 (FIG. 4). These data indicated that expression of TNFSF9 or TNFSF18 was not negatively affected by co-expressing either TNFSF9 or TNFSF18 together with NAP.

### Example 4 Co-Expression of TNFSF9 or TNFSF18 Together with NAP Induces Immunogenic Cell Death

Panc01 and MiaPaCa-2 cells (5×10⁵ cells) were transduced with the different viruses at MOI=50 and plated in 48-well plates. Since transgenes were confirmed in previous examples, here after we only tested oncolytic viruses with transgene TNFSF9 or TFNSF18 co-expressed with NAP, i.e., Ad(O9B) and Ad(O18B). Immunogenic cell death (ICD) was examined 48 hours after transduction by looking at the cell surface calreticulin (CRT) levels by flow cytometry, and adenosine triphosphate (ATP) release in the supernatant using the ATP determination kit (Invitrogen).

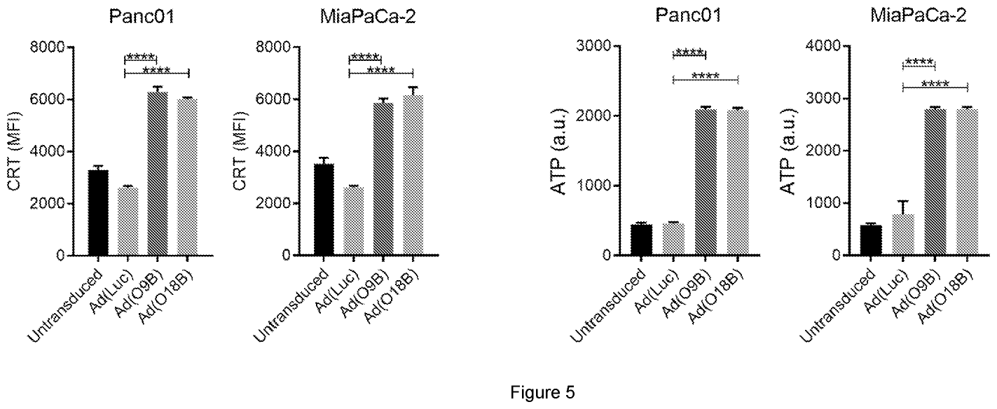

The non-oncolytic control virus Ad(Luc) did not induce any features of ICD, as compared to untreated cells, neither CRT level nor ATP release were increased. On the other hand, all cells transduced with oncolytic virus exhibited high level of CRT and released high level of ATP (FIG. 5), which indicated that ICD had occurred in the transduced cells.

### Example 5 Co-Expression of TNFSF9 or TNFSF18 Together with NAP Induced Dendritic Cell Maturation

Panc01 and MiaPaCa-2 cells (5×10⁵) were transduced with the different viruses at MOI=50 and plated in 48-well plates. 48 hours after transduction, immature dendritic cells (DCs) from different donors were added to the co-culture and 18 hours later DC maturation was examined by measuring surface level expression of DC activation markers (cluster of differentiation 80 (CD80), CD40, CD86, C-C chemokine receptor type 7 (CCR7)). Results represent the mean of three independent experiments.

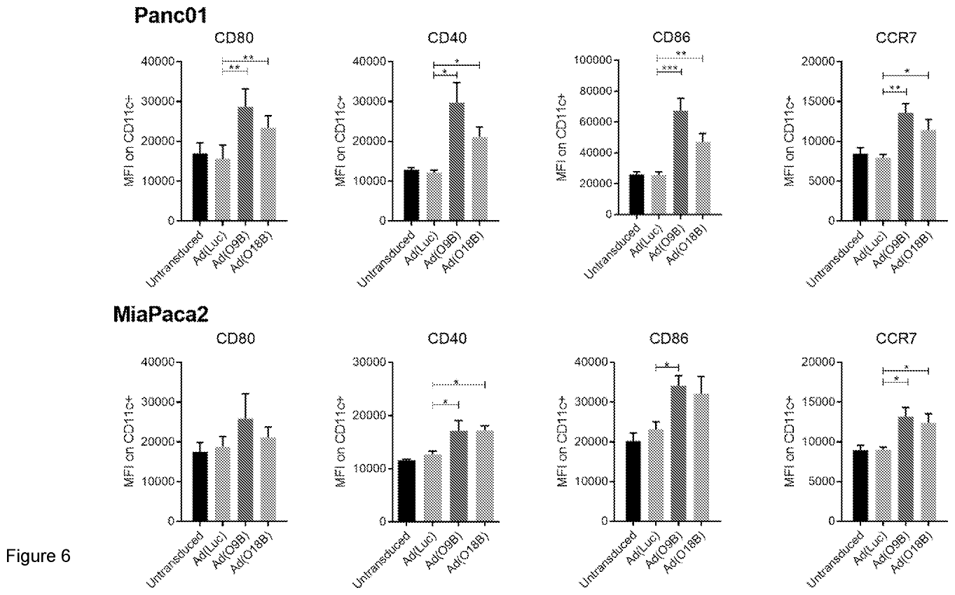

Cells transduced with recombinant oncolytic virus Ad(O9B) or Ad(O18B) were able to mature and activate DCs in the co-culture as indicated by elevated surface expression of maturation and activation markers (CD80, CD40, CD86, CCR7) as presented in FIG. 6. Co-expression of transgene TNFSF9 or TNFSF18 together with NAP was able to induce DC maturation and activation.

### Example 6 Oncolytic Virus Co-Expressing TNFSF9 or TNFSF18 Together with NAP Induced T and NK Cell Activation and Exerted Cytotoxic Function

Female 6-8-week-old C57BI/6 mice (Taconic, Silkeborg, Demark) were subcutaneously (s.c.) implanted with Panc02 cells (1×10⁶ cells in 100 μl Dulbecco's phosphate-buffered saline, (DPBS)) in the right hind flank. The mice were treated intratumorally (i.t.) with PBS (50 μl), or various viruses (1×10¹¹ virus particles (VP) in 50 μl PBS) at day 12 post tumor inoculation when the tumors were palpable (size appox. 50 mm³). Both tumor infiltrating CD8 and CD4 T-cells and NK cells activation were examined three days after treatment. In addition, splenocyte were isolated after virus treatment and mixed with Panc02, IFN-γ release in the supernatant was determined.

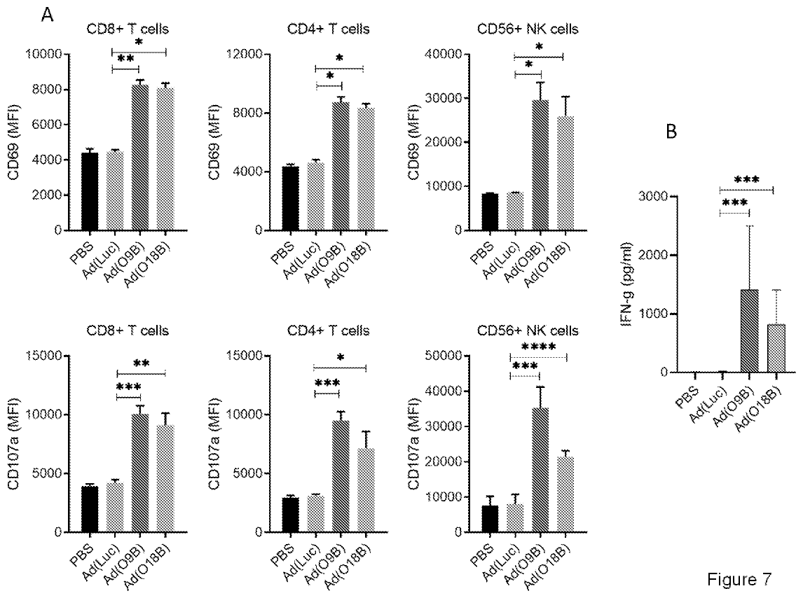

The control virus treatment did not activate T cells or NK cells. On the other hand, both Ad(09B) and Ad(O18B) treatment led to T cell and NK cell activation as shown by upregulation of the surface makers CD69 and CD107a (FIG. 7A). In addition, Ad(O9B) and Ad(O18B) treatment also led to generation of memory as indicated by endogenous splenocytes also reacted with tumor cells by release of significant amount of IFN-γ (FIG. 7B).

### Example 7 Oncolytic Virus Co-Expressing TNFSF9 or TNFSF18 Together with NAP had Enhanced Therapeutic Effect

Female 6-8-week-old C57BI/6 mice (Taconic, Silkeborg, Demark) were subcutaneously (s.c.) implanted with Panc02 cells (1×10⁶ cells in 100 μl DPBS) in the right hind flank. The mice were treated intratumorally (i.t.) with PBS (50 μl), or various viruses (1×10¹¹ VP in 50 μl PBS) at day 7, 10, and 12 post tumor inoculation when the tumors were palpable (size approximately 50 mm³).

In another set of experiment, female 6-8-week-old Athymic-nude mice (JANVIER Labs, France) were subcutaneously (s.c.) implanted with Panc01 cells (5×10⁶ cells in 100 μl 1:1 mixture of DPBS and Matrigel) in the right hind flank. The mice were treated intratumorally (i.t.) with PBS (50 μl), or various viruses (1×10¹¹ VP in 50 μl PBS) at day 7, 10, and 12 post tumor inoculation when the tumors were palpable (size approximately 50 mm³).

The animals were monitored individually for tumor growth until the tumor volume exceeded the study endpoint volume (EPV, 1000 mm³). Tumor size was calculated using the ellipsoid volume formula: tumor volume=(Length×Width²×π)/6.

The time to endpoint (TTE) for each mouse was calculated as TTE=[log(EPV)—b]/m, where the constant b is the intercept and m is the slope of the line obtained by linear regression of time. A log-transformed tumor growth data set, which consisted of the first measured tumor volume when EPV was exceeded and three consecutive measured tumor volumes immediately prior to the attainment of EPV. Survival curve was generated based on the TTE values using the Kaplan-Meier method, and compared using the log-rank (Mantel-Cox) test.

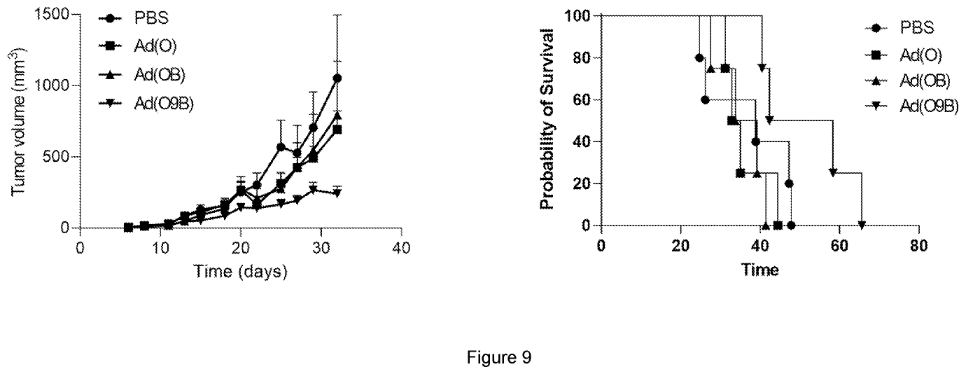

In both the Panc02 model and Panc01 xenograft model, oncolytic viruses Ad(O) and Ad(OB) could marginally inhibit the tumor growth and slightly prolonged the mice survival (FIGS. 8, 9). On the other hand, Ad(09B) and Ad(018B) (only in Panc02 model) significantly delayed the tumor growth and prolonged the mice survival (FIGS. 8, 9). The data from the Panc02 model indicated that co-expression of TNFSF9 or TNFSF18 together with NAP led to significant activation of host immune response and achieved therapeutic anti-tumor effects. The data from Panc01 model indicated that co-expression of TNFSF9 together with NAP led to strong anti-tumor effects, especially adenovirus replication is permissive in this human xenograft model.

### Example 8 Oncolytic Virus Co-Expressing Immunogen GD2-Mimotope Together with NAP Did not Lead to Enhance Therapeutic Effect

Female 6-8-week-old A/J mice (Envigo, The Netherlands) were subcutaneously (s.c.) implanted with NXS2 cells (1×10⁶ cells in 100 μl DPBS) in the right hind flank. The mice were treated intratumorally (i.v.) with PBS (50 μl), or various engineer oncolytic Semliki Forest viruses (1×10¹¹ VP in 50 μl PBS) at day 7 post tumor inoculation when the tumors were palpable (size approximately 50 mm³).

The animal monitoring, measurement of tumor size, and generation of survival curve were as described in Example 7.

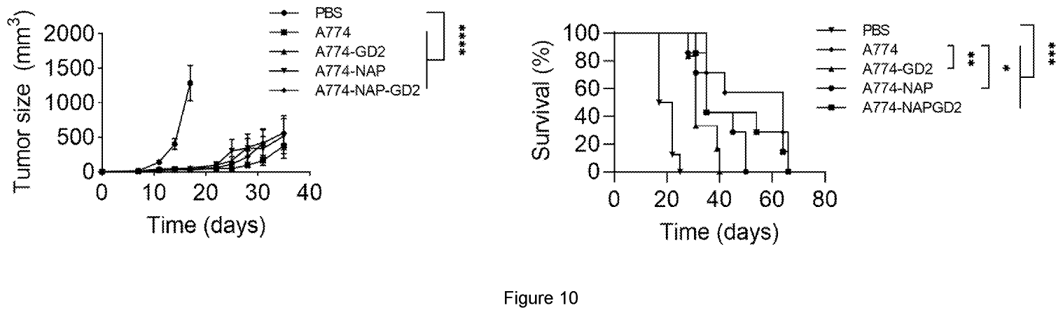

All oncolytic viruses significantly inhibited tumor growth and prolonged the mice survival (FIG. 10). On the other hand, co-expressing the immunogen GD2-mimotope together with NAP in A774-NAPGD2 neither enhanced tumor growth inhibition, nor prolonged survival of these mice (FIG. 10) as compared with oncolytic virus expressing a single factor (GD2 or NAP). These data indicated that synergistic effect as obtained by co-expressing NAP together with an immunomodulating factor is not taking place when co-expressing NAP with an immunogen.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible.

1.-25. (canceled)

26. An adenovirus comprising:
a nucleic acid sequence encoding a Helicobacter pylori neutrophil-activating protein (NAP) and/or a nucleic acid sequence encoding an immunologically equivalent fragment of NAP, wherein the immunologically equivalent fragment of NAP is a fragment including at least one polypeptide domain of at least 20 amino acid residues of NAP; and
a nucleic acid sequence encoding an immunomodulator capable of inducing an immune response in a subject.

27. The adenovirus according to claim 26, wherein the immunomodulator is capable of inducing dendritic cell (DC) maturation, T cell activation and/or NK cell activation in the subject.

28. The adenovirus according to claim 27, wherein the immunomodulator is capable of inducing DC maturation, T cell activation and NK cell activation in the subject.

29. The adenovirus according to claim 27, wherein the immunomodulator is capable of inducing dendritic cells to express cluster of differentiation 80 (CD80), CD40, CD86 and C-C chemokine receptor type 7 (CCR7).

30. The adenovirus according to claim 27, wherein the immunomodulator is capable of inducing CD4+ T cell activation and/or CD8+ T cell activation.

31. The adenovirus according to claim 30, the immunomodulator is capable of inducing CD4+ T cells and/or CD8+ T cells to express cluster of differentiation 69 (CD69) and CD107a.

32. The adenovirus according to claim 27, wherein the immunomodulator is capable of inducing CD56+NK cell activation.

33. The adenovirus according to claim 32, the immunomodulator is capable of inducing CD56+NK cells to express cluster of differentiation 69 (CD69) and CD107a.

34. The adenovirus according to claim 26, wherein the immunomodulator is a tumor necrosis factor superfamily (TNFSF) member.

35. The adenovirus according to claim 34, wherein the TNFSF member is selected from the group consisting of TNFSF1, TNFSF2, TNFSF4, TNFSF5, TNFSF7, TNFSF9, TNFSF14, TNFSF18 and a combination thereof.

36. The adenovirus according to claim 35, wherein the TNFSF member is selected from the group consisting of TNFSF5, TNFSF9, TNFSF14, TNFSF18 and a combination thereof.

37. The adenovirus according to claim 36, wherein the TNFSF member is selected from the group consisting of TNFSF9, TNFSF18 and a combination thereof.

38. The adenovirus according to claim 26, wherein the immunologically equivalent fragment of NAP is a fragment including at least one polypeptide domain of at least 30 amino acid residues of NAP.

39. The adenovirus according to claim 26, wherein the immunologically equivalent fragment of NAP is selected from the group consisting of SEQ ID NO: 11 to 14.

40. The adenovirus according to claim 26, further comprising a nucleic acid sequence encoding a self-cleaving peptide positioned between the nucleic acid sequence encoding NAP, and/or the immunologically equivalent fragment of NAP, and the nucleic acid sequence encoding the immunomodulator.

41. The adenovirus according to claim 40, wherein the self-cleaving peptide is selected from the group consisting of SEQ ID NO: 16 to 23.

42. The adenovirus according to claim 26, wherein the adenovirus is an oncolytic adenovirus.

43. The adenovirus according to claim 42, wherein the oncolytic adenovirus comprises a mutated adenovirus early region 1A (EIA) gene encoding a mutated EIA protein having a lower Rb protein binding capability as compared to a wild-type EIA protein.

44. The adenovirus according to claim 43, wherein the mutated ELA gene comprises a 24 bp deletion of nucleotides 919 to 943 of wild-type EIA gene.

45. The adenovirus according to claim 43, wherein the mutated EIA protein lacks amino acids 121 to 128 of wild-type EIA protein.

46. The adenovirus according to claim 26, wherein
one of the nucleic acid sequences encoding 19-kDa adenovirus E1B protein and 55-kDa adenovirus EIB protein is replaced by the nucleic acid sequence encoding NAP and/or the nucleic acid sequence encoding the immunologically equivalent fragment of NAP; and
the other of the nucleic acid sequences encoding 19-kDa adenovirus E1B protein and 55-kDa adenovirus E1B protein is replaced by the nucleic acid sequence encoding the immunomodulator.

47. The adenovirus according to claim 26, wherein the adenovirus is a human adenovirus type 5.

48. A method for treatment of cancer comprising administering an effective amount of an adenovirus according to claim 26 to a subject suffering from cancer.

49. The method according to claim 48, wherein the cancer is selected from the group consisting of carcinoma, sarcoma, lymphoma, leukemia, seminoma, germinoma, dysgerminoma and blastoma.

50. The method according to claim 48, wherein the cancer is selected from the group consisting of pancreatic cancer, breast cancer, lung cancer, liver cancer, kidney cancer; osteosarcoma, liposarcoma, non-Hodgkin lymphoma, Hodgkin lymphoma, acute leukemia, chronic leukemia, glioblastoma and neuroblastoma.


SEQUENCE LISTING (25 sequences)

SEQ ID NO: 1 - DNA, 21 bp, Artificial Sequence
  Forward PCR primer
catcaggttgattcacatcgg

SEQ ID NO: 2 - DNA, 21 bp, Artificial Sequence
  Reverse PCR primer
gaagcgctgtatgttgttctg

SEQ ID NO: 3 - protein, 144 aa, Helicobacter pylori
MKTFEILKHLQADAIVLFMKVHNFHWNVKGTDFFNVHKATEEIYEEFADMFDDLAERIVQLGHHPLVTLSEAIKLTRVKEETKTSFHSKDIFKEILEDYKYLEKEFKELSNTAEKEGDKVTVTYADDQLAKLQKSIWMLQAHLA

SEQ ID NO: 4 - protein, 144 aa, Helicobacter pylori
MKTFEILKHLQADAIVLFMKVHNFHWNVKGTDFFNVHKATEEIYEEFADMFDDLAERIVQLGHHPLVTLSEAIKLTRVKEETKTSFHSKDIFKEILEDYKHLEKEFKELSNTAEKEGDKVTVTYADDQLAKLQKSIWMLQAHLA

SEQ ID NO: 5 - protein, 144 aa, Helicobacter pylori
MKTFEILKHLQADAIVLFMKVHNFHWNVKGTDFFNVHKATEEIYEEFADMFDDLAERIVQLGHHPLVTLSEALKLTRVKEETKTSFHSKDIFKEILEDYKYLEKEFKELSNTAEKEGDKVTVTYADDQLAKLQKSIWMLQAHLA

SEQ ID NO: 6 - protein, 144 aa, Helicobacter pylori
MKTFEILKHLQADAIVLFMKVHNFHWNVKGTDFFNVHKATEEIYEEFADMFDDLAERIVQLGHHPLVTLSEALKLTRVKEETKTSFHSKDIFKEILEDYKHLEKEFKELSNTAEKEGDKVTVTYADDQLAKLQKSIWMLQAHLA

SEQ ID NO: 7 - protein, 144 aa, Helicobacter pylori
MKTFEILKHLQADAIVLFMKVHNFHWNVKGTDFFNVHKATEEIYEGFADMFDDLAERIVQLGHHPLVTLSEAIKLTRVKEETKTSFHSKDIFKEILEDYKYLEKEFKELSNTAEKEGDKVTVTYADDQLAKLQKSIWMLQAHLA

SEQ ID NO: 8 - protein, 144 aa, Helicobacter pylori
MKTFEILKHLQADAIVLFMKVHNFHWNVKGTDFFNVHKATEEIYEGFADMFDDLAERIVQLGHHPLVTLSEALKLTRVKEETKTSFHSKDIFKEILEDYKYLEKEFKELSNTAEKEGDKVTVTYADDQLAKLQKSIWMLQAHLA

SEQ ID NO: 9 - protein, 144 aa, Helicobacter pylori
MKTFEILKHLQADAIVLFMKVHNFHWNVKGTDFFNVHKATEEIYEGFADMFDDLAERIVQLGHHPLVTLSEAIKLTRVKEETKTSFHSKDIFKEILEDYKHLEKEFKELSNTAEKEGDKVTVTYADDQLAKLQKSIWMLQAHLA

SEQ ID NO: 10 - protein, 144 aa, Helicobacter pylori
MKTFEILKHLQADAIVLFMKVHNFHWNVKGTDFFNVHKATEEIYEGFADMFDDLAERIVQLGHHPLVTLSEALKLTRVKEETKTSFHSKDIFKEILEDYKHLEKEFKELSNTAEKEGDKVTVTYADDQLAKLQKSIWMLQAHLA

SEQ ID NO: 11 - protein, 36 aa, Helicobacter pylori
EILKHLQADAIVLFMKVHNFHWNVKGTDFFNVHKAT

SEQ ID NO: 12 - protein, 34 aa, Helicobacter pylori
NTAEKEGDKVTVTYADDQLAKLQKSIWMLQAHLA

SEQ ID NO: 13 - protein, 36 aa, Helicobacter pylori
ATEEIYEEFADMFDDLAERIVQLGHHPLVTLSEALK

SEQ ID NO: 14 - protein, 36 aa, Helicobacter pylori
LTRVKEETKTSFHSKDIFKEILEDYKHLEKEFKELS

SEQ ID NO: 15 - protein, 8 aa, Artificial Sequence
  2A core motif
  misc_feature (2)..(2): Xaa can be any naturally occurring amino acid
  misc_feature (4)..(4): Xaa can be any naturally occurring amino acid
DXEXNPGP

SEQ ID NO: 16 - protein, 18 aa, Thosea asigna virus
EGRGSLLTCGDVEENPGP

SEQ ID NO: 17 - protein, 21 aa, Artificial Sequence
  Self-cleaving 2A peptide
GSGEGRGSLLTCGDVEENPGP

SEQ ID NO: 18 - protein, 19 aa, Porcine teschovirus-1
ATNFSLLKQAGDVEENPGP

SEQ ID NO: 19 - protein, 22 aa, Artificial Sequence
  Self-cleaving 2A peptide
GSGATNFSLLKQAGDVEENPGP

SEQ ID NO: 20 - protein, 20 aa, Equine rhinitis A virus
QCTNYALLKLAGDVESNPGP

SEQ ID NO: 21 - protein, 23 aa, Artificial Sequence
  Self-cleaving 2A peptide
GSGQCTNYALLKLAGDVESNPGP

SEQ ID NO: 22 - protein, 22 aa, Foot-and-mouth disease virus
VKQTLNFDLLKLAGDVESNPGP

SEQ ID NO: 23 - protein, 25 aa, Artificial Sequence
  Self-cleaving 2A peptide
GSGVKQTLNFDLLKLAGDVESNPGP

SEQ ID NO: 24 - DNA, 24 bp, Adenovirus type 5
cttacctgccaggaggctggcttt

SEQ ID NO: 25 - protein, 8 aa, Adenovirus type 5
LTCHEACF